(19) United States
(12) Patent Application Publication
(10) Pub. No.: US 2017/0065748 A1
(43) Pub. Date: Mar. 9, 2017

(54) SURFACE MODIFICATION METHOD AND STRUCTURE FOR IMPROVING HEMOCOMPATIBILITY OF BIOMEDICAL METALLIC SUBSTRATES

(71) Applicant: NATIONAL CHUNG-HSING UNIVERSITY, Taichung City (TW)

(72) Inventors: Shu-Ping Lin, Taichung City (TW); Pei-Chieh Wong, Taichung City (TW)

(21) Appl. No.: 15/256,751

(22) Filed: Sep. 6, 2016

(30) Foreign Application Priority Data
Sep. 9, 2015 (TW) 104129830

(51) Int. Cl. A61L 31/08; A61F 2/82; A61F 2/06; A61L 31/02

(52) U.S. Cl. CPC A61L 31/08; A61L 31/088; A61L 31/022; A61F 2/82; A61F 2/06; A61L 2420/08; A61L 2420/02; A61L 2400/18; A61L 2300/608; A61F 2310/0061; A61F 2310/00916; A61F 2310/00856; A61F 2240/001

(57) ABSTRACT

The present invention relates to a surface modification method for improving the hemocompatibility of biomedical metallic substrate, comprising: fixing a sulfur-containing monomolecular film on the surface of oxide layer of a biomedical metallic substrate by molecular self-assembly. The surface modification will improve the hydrophilicity and hemocompatibility of the biomedical metallic substrate in contact with the blood, and ensure that the biomedical metallic substrate is non-toxic to the endothelial cells.

### CROSS-REFERENCE TO RELATED APPLICATIONS

The present application claims benefit of priority of the Taiwan Patent Application No. 104129830, filed Set. 9, 2015. The entirety of said Taiwan application is incorporated by reference herein.

### STATEMENT REGARDING PRIOR DISCLOSURES BY THE INVENTOR

The subject matter of the present invention was previously disclosed in the Master's Thesis entitled “TiO₂-nanotube Surface for investigation of Biocompatibility and Hemocompatibility”, by Pei-Chieh Wong under the guidance of Prof. Shu-Ping Lin, presented Jul. 28, 2015, at National Chung-Hsing University, Taichung, Taiwan. Both Pei-Chieh Lin and Shu-Ping Lin are named joint inventors of the present application. The said Master's Thesis, a copy of which is being submitted as attachment to the present application, is a grace period inventor disclosure under 35 U.S.C. 102(b)(1)(A).

### BACKGROUND OF THE INVENTION

Technical Field of the Invention

The present invention relates to a surface modification technique for improving the biocompatibility, especially the hemocompatibility, of biomedical metallic substrates. Particularly, the present invention relates to modification of sulfur-containing silanol functional group on the surface of metallic materials.

Background

The known biomedical metallic materials include platinum, gold, tungsten, rhenium, palladium, rhodium, ruthenium, titanium, nickel, iridium and alloys of these metals, such as stainless steel, titanium/nickel alloy, and platinum/iridium alloy. Such metallic materials can be made into the implant for contact with living tissue or long-term exposure to the blood, and can also be used as a surface coating material to other substrates. The implant for exposing to the blood should possess superior hemocompatibility.

In the case of vascular stents, the bare metallic stent is typically made of 316L stainless steel, cobalt-based alloys, titanium or tantalum. The drug-eluting stents are coated with drug-containing coating on the surface of the metal stent, for sustained release of the drugs in the coating to the bloodstream. The coating may be a polymeric coating, such as polyethylene-co-vinyl acetate (PEVA), poly n-butyl methacrylate (PBMA), and the like. The drug contained in the coating may be an anticoagulant, such as heparin, or a drug inhibiting smooth muscle cell growth, such as sirolimus and paclitaxel (Taxol).

WO2014169281 discloses a vascular stent coated with polyelectrolyte multilayers of a polycation and a polyanion. The polycation may be chitosan. The polyanion may be a glycosaminoglycan. At least one of the polycation or polyanion may include nitric oxide-releasing groups. The medical device may release nitric oxide from the surface for the purpose of reducing platelet activation. The medical device may further include a growth factor adsorbed on at least one of the polyelectrolyte layer. The growth factor may be vascular endothelial growth factor (VEGF).

US20130224795 discloses a method for immobilizing a bioactive molecule onto a substrate surface by using polyphenol oxidase. In the presence of polyphenol oxidase, a bioactive molecule containing a phenol or catechol group can be simply in situ oxidized within a short time to dopa or dopaquinone which forms a coordinate bond with a metal or polymer substrate, thus stably immobilizing the bioactive molecule onto the substrate surface. The bioactive molecules include cell adhesion peptides, growth factors, growth hormones, proteins, anti-thrombotic agents, and endothelialization inducing agents. The cell adhesion peptides and growth factors can be simply immobilized to medical metal or polymer substrate surfaces such as orthopedic or dental implants. Also, antithrombotic agents and/or endothelialization inducing agents may be immobilized to medical substrates for vascular systems, such as stents and artificial blood vessels.

Coating technology has been widely used in the surface modification of biomedical substrates. However, the coated film is physically attached to the surface of a biomedical substrate, and the stability of physical binding is relatively weaker than the immobilization of bioactive molecules. As for the immobilization technique of bioactive molecules, its disadvantages are the technical complexity and taking a long period of time to achieve the chemical reactions for the immobilization. Additionally, it is difficult to remove or control the production and side effects of the byproducts from the bioactive molecules.

### SUMMARY OF INVENTION

The purpose of the invention is to provide a surface modification method for improving the hemocompatibility of biomedical metallic substrate, comprising forming a sulfur-containing monomolecular film on the surface of oxide layer of biomedical metallic substrate by molecular self-assembly. The surface modification will improve the hydrophilicity and hemocompatibility of the biomedical metallic substrate in contact with the blood, and ensure that the biomedical metallic substrate is non-toxic to the endothelial cells.

The surface modification method for improving the hemocompatibility of biomedical metallic substrate comprises: immobilizing a sulfur-containing monomolecular film on the surface of oxide layer of biomedical metallic substrate by molecular self-assembly.

In certain embodiments of the invention, the biomedical metal is preferably a titanium or titanium alloy. The oxide layer may be a native oxide or an oxide layer created by a surface modification technique. The molecular self-assembly comprises: contacting the biomedical metallic substrate having an oxide layer with a solution of a silanol chemical derivative containing mercapto group or sulfur atom for a predetermined period of time, and immobilizing a sulfur-containing monomolecular film exposing functional mercapto group or sulfur atom on the surface of the oxide layer by self-assembly.

The modification of the surface of oxide layer of biomedical metallic substrate with a sulfur-containing monomolecular film will confer hydrophilic and hemocompatible properties to the substrate. As used herein, “hemocompatibility” refers to the blood clotting time after contacting with the biomedical metallic substrate, including prothrombin time (PT) and activated partial thromboplastin time (aPTT), being in a normal range, and lowered fibrinogen concentration of the contacting substrate surface. In addition, there is no platelet activation occurring in the blood contacting the substrate surface, and the substrate is non-toxic to the endothelial cells.

“PT and aPTT are in a normal range” means the biomedical metallic substrate of the present invention does not adversely affect the exogenous coagulation pathway and endogenous coagulation pathway of blood, and can maintain the dynamic equilibrium between blood coagulation and anti-coagulation.

Fibrinogen is a glycoprotein in vertebrates that helps in the formation of blood clots. Thromboplastin is released from damaged platelets, and converts prothrombin to thrombin in the action of calcium ion. Thrombin coagulates the originally water-soluble fibrinogen into water-insoluble fibrin. Fibrin links other blood cells into aggregation and becomes solidified blood clot. The substrate of the present invention would reduce the fibrinogen concentration in the contacted blood, so that the fibrin cannot be produced to entangle blood cells, and further ensure that no blood clots are formed on the substrate surface. Therefore, the expected physiological effect of lowering fibrinogen concentration is to prevent the formation of blood clot.

The platelet activation will prime greater blood coagulation. According to the present invention, platelet activation will not occur in the blood that contacts with the substrate surface, which ensures that no blood clots are formed on the substrate surface.

Erythrocyte adsorption easily leads to the abnormal aggregation of blood cells, which becomes the base for thrombosis. No erythrocyte adsorption occurs on the substrate surface of present invention. The expected physiological effect is not inducing thrombus formation.

Vascular endothelial cells ensure the integrity of vascular wall, and can promote natural healing of the vascular wall. The incomplete or delayed healing of vascular endothelium will result in the highly exposed extracellular matrix, which activates the coagulation and leads to thrombosis. The substrate of present invention is totally non-toxic to the endothelial cells, and allows endothelial cells normally grow on the substrate surface. The expected physiological effects do not damage the endothelial cells and not activate the coagulation and thrombosis.

The surface modification method for improving the hemocompatibility of biomedical metallic substrate further comprises forming a nitric oxide (NO) layer on the sulfur-containing monomolecular film that is immobilized on the surface of the biomedical metallic substrate.

The surface modification method for improving the hemocompatibility of biomedical metallic substrate further comprises forming a nitric oxide (NO) layer on the surface of oxide layer of a biomedical metallic substrate, and forming a sulfur-containing monomolecular film on the surface of the nitric oxide (NO) layer by molecular self-assembly.

By the addition of the nitric oxide (NO) layer, the occurrence of platelet activation and blood cell adhesion on the substrate of present invention will be reduced.

The present invention provides the formation of a sulfur-containing monomolecular film on the surface of a biomedical metallic substrate through molecular self-assembly. Relative to the active molecule fixing technology, the method of the present invention requires a short reaction time, is easy to operate, and does not produce any by-product that is difficult to remove or control.

### BRIEF DESCRIPTION OF THE DRAWINGS

FIG. 1A illustrates the chemical structure of a titanium dioxide substrate surface modified with a monomolecular film consisting of sulfur-containing silanol functional group. FIG. 1B illustrates the chemical structure of a titanium dioxide substrate surface modified with a nitric oxide (NO) layer and a monomolecular film consisting of sulfur-containing silanol functional group.

FIG. 2 shows the ESCA analyses of S_{2p3/2} scan for the four samples described in the Example.

FIG. 3 shows the results of hydrophilicity evaluation of the four samples described in the Example by droplet angle goniometry.

FIG. 4 shows the Field-emission scanning electron microscope (FESEM) graph of the platelet-adsorbed surface of the four samples described in the Example.

FIG. 5 shows the FESEM graph of the erythrocyte-adsorbed surface of the four samples described in the Example.

FIG. 6 shows the fluorescent staining of endothelial cells cultured on the surface of the four samples described in the Example.

FIG. 7 shows the quantitative analyses of endothelial cell numbers cultured on the surface of the four samples described in the Example.

### DETAILED DESCRIPTION OF THE INVENTION

The characteristics and advantages of the present invention will be further illustrated and described in the following examples. The examples described herein are used for illustrations, not for limitations of the invention.

The surface modification of the present invention for improving the hemocompatibility of titanium- or titanium alloy-based biomedical metallic substrate comprises: forming a sulfur-containing monomolecular film on the surface of oxide layer of a biomedical metallic substrate by molecular self-assembly.

The said oxide layer may be a native oxide or an oxide layer created by a surface modification technique. In the present invention, the oxide layer (—Ti—O—) is created on the surface of a titanium or titanium alloy substrate (referred to as a titanium dioxide substrate) by an anode oxidation process or gas plasma surface treatment. The oxide layer provides the chemical bonding required in the subsequent molecular self-assembly.

In certain embodiments of the present invention, the steps of said molecular self-assembly comprise: immersing the titanium dioxide substrate in a 0.1%˜20% solution of a silanol chemical derivative containg mercapto group for a period of 10 minutes to 24 hours. During the immersion, the molecular self-assembly is performed on said oxide layer through silanol group, and the sulfur-containing functional groups are then exposed on the outermost surface of the metals.

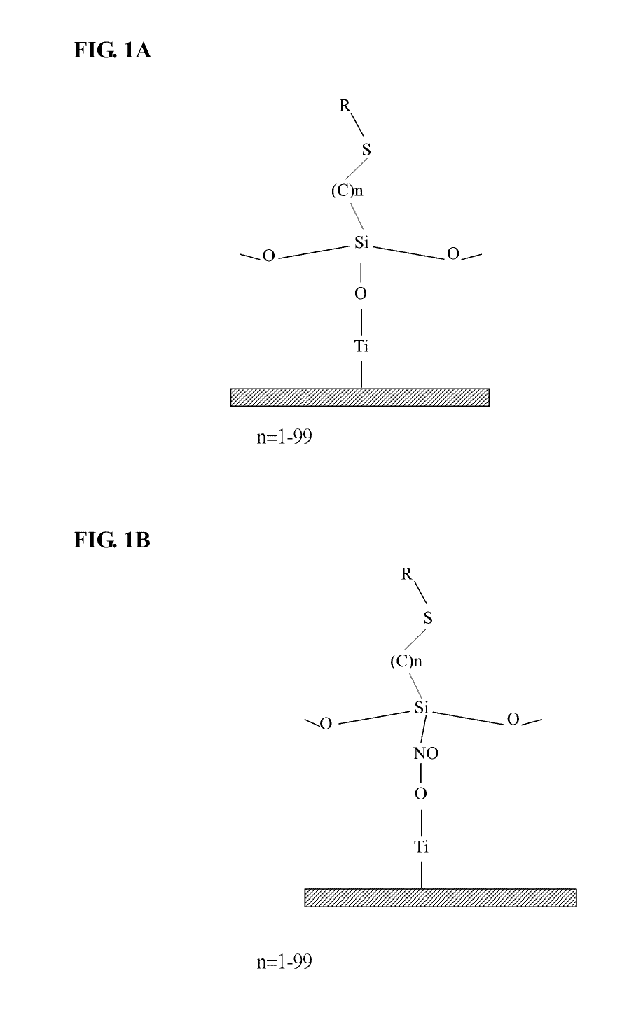

As shown in FIG. 1, the sulfur-containing functional group (—SH) was immobilized on the surface of titanium dioxide substrate by the silanol group binding with titanium (—Ti—O—Si—).

Additionally, a further nitric oxide (NO) layer is formed on the surface of the sulfur-containing monomolecular film by plasma treatment. Alternately, the nitric oxide (NO) layer is formed on the surface of titanium dioxide substrate, and a sulfur-containing monomolecular film is formed on the surface of the nitric oxide (NO) layer by molecular self-assembly.

To confirm the hemocompatibility of the surface modified substrate as described, four samples were prepared to carry out the related measurement and analysis. In the following, MPTMS stands for 3-mercaptopropyltrimethoxysilane. The four samples include: A, titanium substrate (Ti); B, titanium dioxide substrate modified with MPTMS (MPTMS-ATN), with a chemical structure as shown in FIG. 1A; C, titanium dioxide substrate modified with NO-coated MPTMS (NO-MPTMS-ATN); and D, NO-coated titanium dioxide substrate modified with MPTMS (MPTMS-NO-ATN), with a chemical structure as shown in FIG. 1B. The substrate A is a control, and substrates B, C and D are exemplary substrates of present invention.

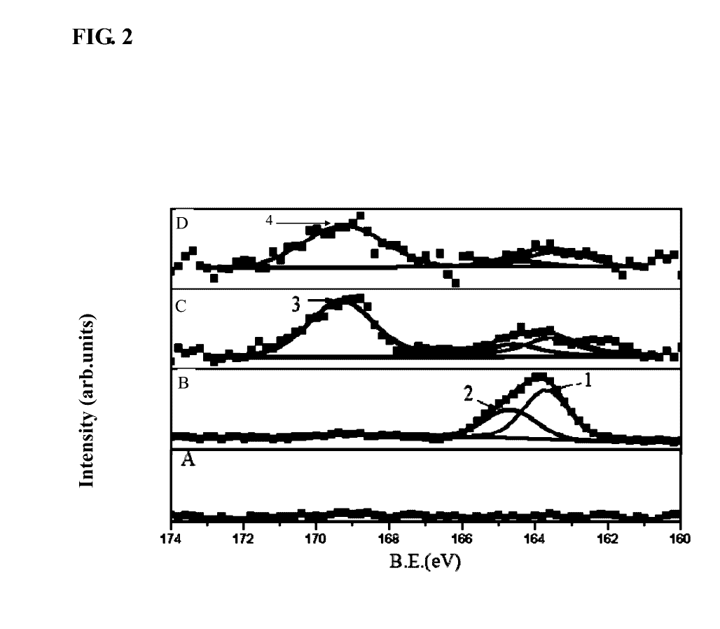

To make sure if the sulfur-containing functional group (—S—) was successfully immobilized on the surface of titanium dioxide substrate, the chemical elements contained in the substrate surface were analyzed by using electron spectroscopy for chemical analysis (ESCA). As shown in FIG. 2, after the scanning of ESCA, S_{2p3/2} scan shows the substrates B, C and D possessed the signals of (1) —SH and —SN— of 163.6 eV; (2) —SH of 164.6 eV; (3) —SO₄²⁻ of 169 eV; (4) —SO₄²⁻ of 169 eV. The substrate A showed no S signal. It is proven that sulfur-containing functional group has successfully modified the surface of substrates B, C and D.

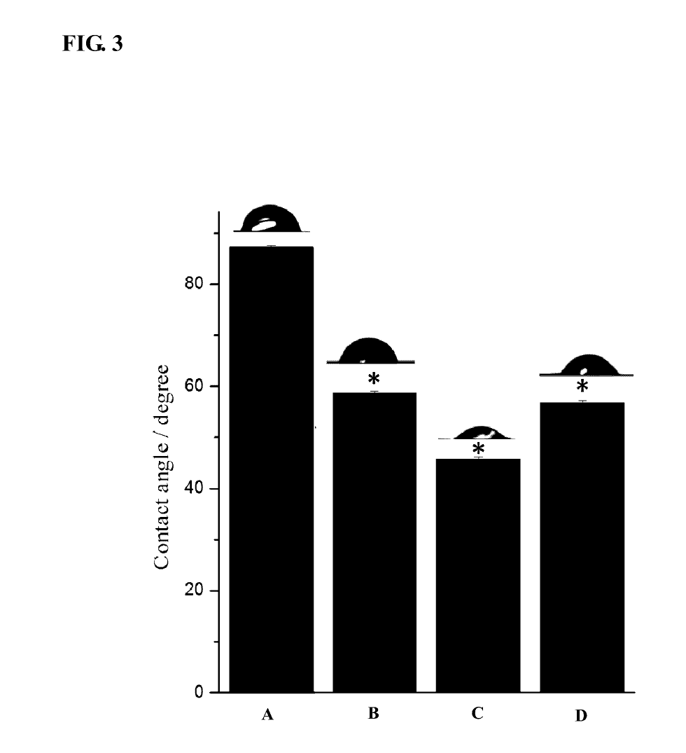

The hydrophilicity of the four samples was evaluated by droplet angle goniometry. Results are shown in FIG. 3. The contact angle of substrate A (Ti) was almost 90°, indicating it was hydrophobic. The contact angles of substrates B (MPTMS-ATN), C (NO-MPTMS-ATN) and D (MPTMS-NO-ATN) were less than 60°, indicating they were relatively hydrophilic.

Blood testing. The four substrates were incubated with fresh blood respectively to investigate the coagulation and anticoagulation actions of the substrates. Platelet-poor plasma (PPP), platelet-rich plasma (PRP) and red blood cells (RBCs) were isolated from fresh blood by centrifugation. The PPP was contacted with the four substrates and incubated at 37° C. in CO₂ incubator for one hour, then subjected to the tests of PT and aPTT, and the measurement of fibrinogen concentration. Furthermore, the four substrates were contacted with PRP and RBC and incubated at 37° C. in CO₂ incubator for one hour, then the adhesion of platelet or blood cell on the surface of four substrates were observed by Field-emmision scanning electron microscope (FESEM).

The results of PT, aPTT and fibrinogen concentration analyses are listed in Table 1. It is shown that the PT and aPTT values of the four substrates are in the normal range, indicating that no negative effects on the exogenous coagulation pathway and endogenous coagulation pathway of blood were produced, and the dynamic equilibrium between blood coagulation and anti-coagulation was maintained. The fibrinogen concentrations on the surface of the four substrate groups were all lower than the normal value, and no blood clotting was observed on the surface of substrates.

TABLE 1

| | | | fibrinogen |
|---|---|---|---|
| | | | concentration |
| | PT(sec) | aPTT(sec) | (mg/dL) |
| Normal range | 8.0~12.0 | 23.9~35.5 | 200.0~400.0 |
| Substrate A (Ti) | 11.08 ± 0.08 | 29.36 ± 0.51 | 195.64 ± 3.28 |
| Substrate B | 11.27 ± 0.4 | 29.23 ± 0.59 | 191.27 ± 5.93 |
| (MPTMS-ATN) | | | |
| Substrate C | 11.3 ± 0.2 | 29.8 ± 0.7 | 189.23 ± 5.03 |
| (NO-MPTMS-ATN) | | | |
| Substrate D | 11.17 ± 0.31 | 29.33 ± 0.31 | 188.67 ± 2.04 |
| (MPTMS-NO-ATN) | | | |

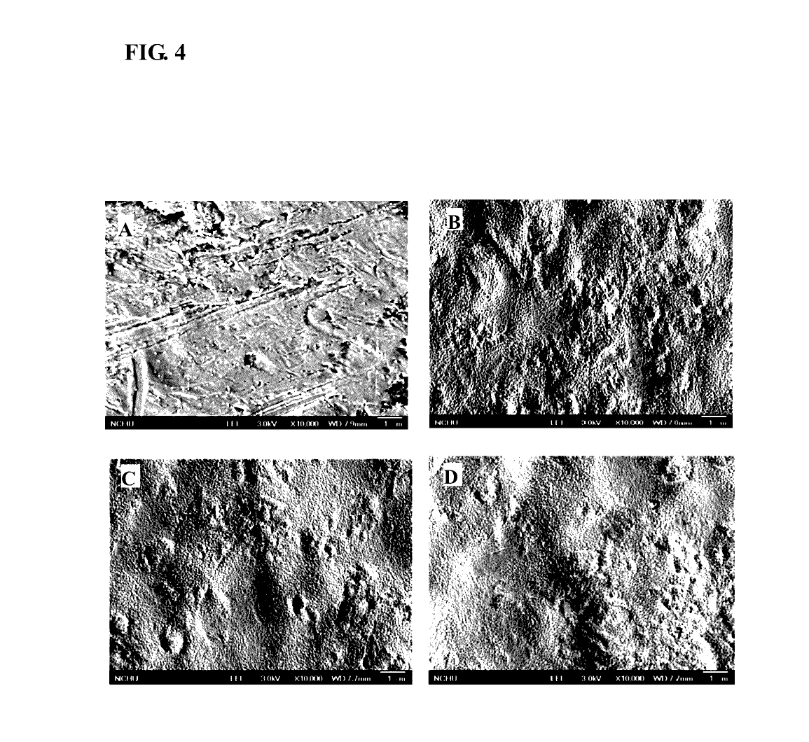

The observed results of platelet adhesion on the surface of the four substrates by FESEM are shown in FIG. 4. Platelets were adsorbed on the substrate A (Ti), while no platelets were adsorbed on the substrates B (MPTMS-ATN), C (NO-MPTMS-ATN) and D (MPTMS-NO-ATN). Platelet activation occurred on the substrate A, but no platelet activation occurred on the substrates B, C and D of the present invention. The platelet activation will prime blood coagulation. Platelet activation did not occur on the surface of substrates B, C and D of the present invention, which ensures that no blood clots are formed on the substrate surface.

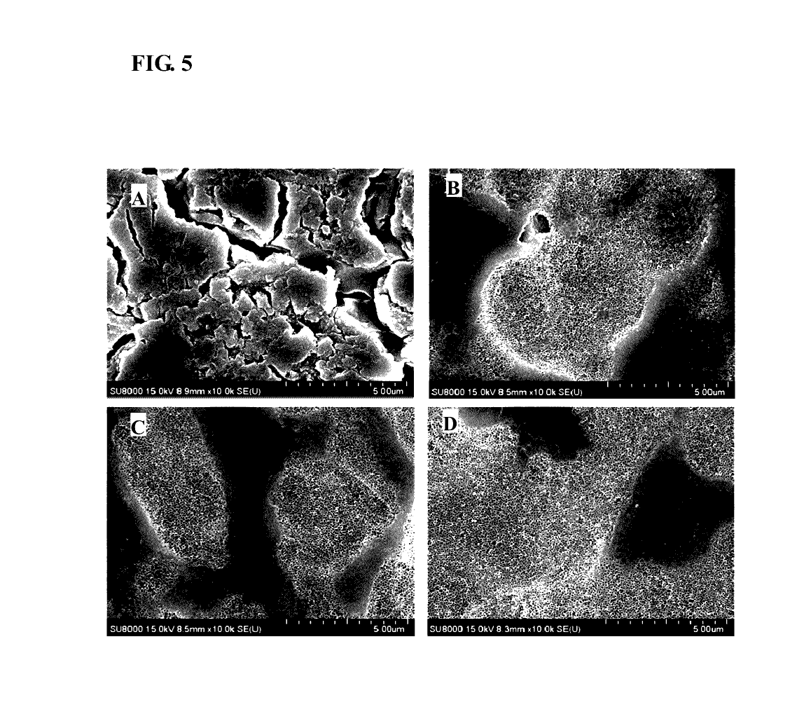

The observed results of red blood cells (RBCs) adhesion on the surface of the four substrates by FESEM are shown in FIG. 5. There was no red blood cell adsorbing on the surface of the four substrates. The expected physiological effect of no RBC adhesion on the surface of present substrates is that no thrombus formation is induced.

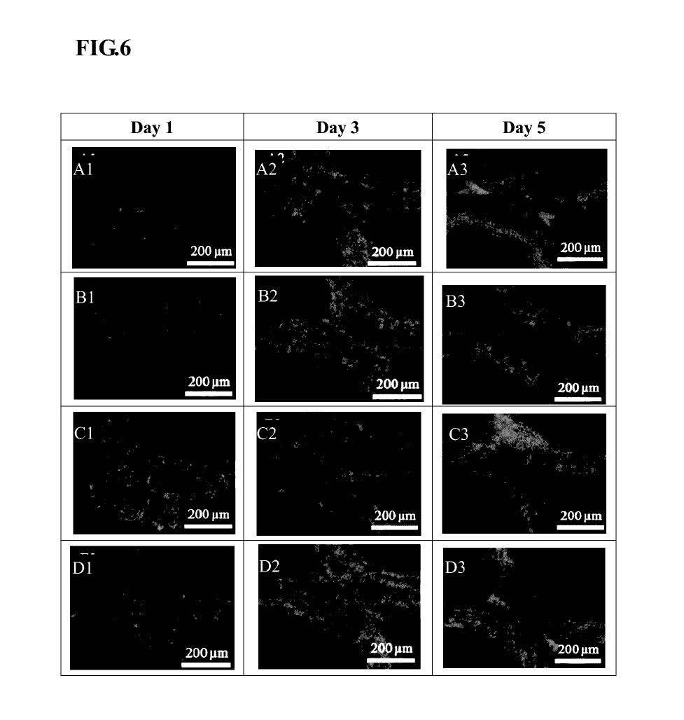

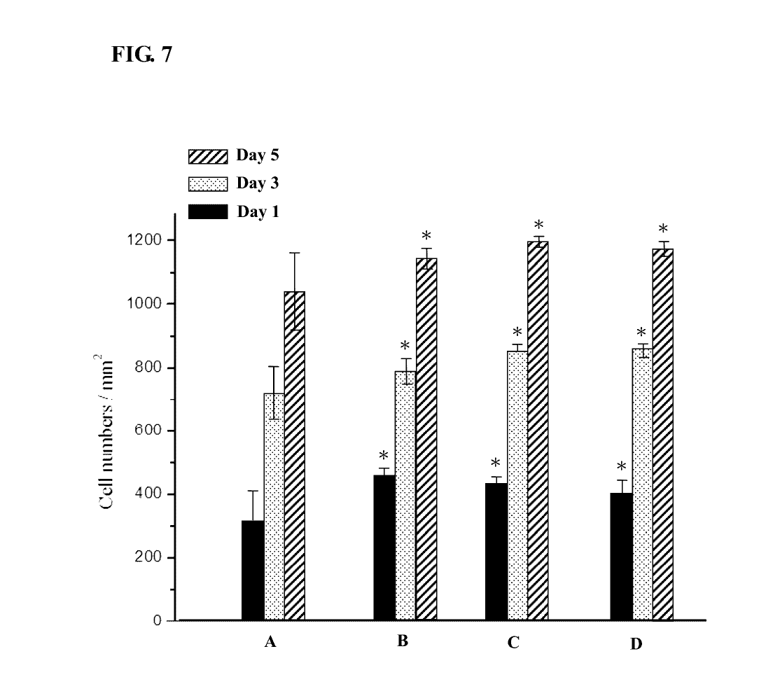

For the vascular endothelium test, vascular endothelial cells were attached to the surface of the four substrates. The nucleus of endothelial cell was stained with the fluorescein dye DAPI, and the staining result was observed using a fluorescence microscope. The growth of endothelial cell on the surface of the four substrates at Day 1, Day 3 and Day 5 are shown in FIG. 6. FIG. 7 shows the statistic analyses of the number of growing endothelial cells. As shown in the FIGS. 6 and 7, the number of endothelial cells increased with the progressive days, especially the cell numbers were greater in the substrate B, C and D groups than in the substrate A. The results indicate that the substrates B, C and D of the present invention were non-toxic to endothelial cells, promising the normal growth and proliferation of endothelial cells on the substrate surface. The expected physiological effect is no activation of the coagulation and thrombosis.

What is claimed is:

1. A surface modified structure of biomedical metallic substrate for improving hemocompatibility, comprising:
a silanol-oxide layer formed on a surface of a biomedical metallic substrate, and
a sulfur-containing monomolecular film with exposed sulfur-containing functional groups formed on the surface of the silanol-oxide layer.

2. The surface modification structure of claim 1, further comprising a nitric oxide (NO) layer formed on the surface of the sulfur-containing monomolecular film.

3. A method for preparing the surface modified structure of claim 1, comprising:
contacting a biomedical metallic substrate having an oxide layer with a solution of a silanol chemical derivative containing mercapto group or sulfur atom for a predetermined period of time to form a silanol-oxide layer on the biomedical metallic substrate, and a sulfur-containing monomolecular film exposing sulfur-containing functional groups on outermost surface of the silanol-oxide layer by means of molecular self-assembly.

4. The method of claim 3, wherein the predetermined period of time is 10 minutes to 24 hours.

5. The method of claim 3, wherein the silanol chemical derivative is 3-mercaptopropyltrimethoxysilane (MPTMS).

6. The method of claim 3, wherein the solution of silanol chemical derivative has a volume concentration of 0.1%˜20%.

7. The method of claim 3, further comprising a step of forming a nitric oxide (NO) layer on the surface of the sulfur-containing monomolecular film.

8. A surface modification method for improving hemocompatibility of biomedical metallic substrate, comprising:
forming a nitric oxide (NO) layer on the surface of an oxide layer of a biomedical metallic substrate,
and forming a sulfur-containing monomolecular film on the surface of the nitric oxide (NO) layer by means of molecular self-assembly.

9. The surface modification method of claim 8, wherein the molecular self-assembly comprises:
contacting the biomedical metallic substrate having the nitric oxide (NO) layer with a solution of a silanol chemical derivatives containing mercapto group or sulfur atom for a predetermined period of time to form a sulfur-containing monomolecular film exposing functional mercapto group or sulfur atom on the surface of the nitric oxide (NO) layer by self-assembly.

10. The surface modification method of claim 9, wherein the predetermined period of time is 10 minutes to 24 hours.

11. The surface modification method of claim 9, wherein the silanol chemical derivative is 3-mercaptopropyltrimethoxysilane (MPTMS).

12. The method of claim 9, wherein the solution of silanol chemical derivative has a volume concentration of 0.1%˜20%.
